(19) United States
(12) Patent Application Publication
(10) Pub. No.: US 2009/0306902 A1
(43) Pub. Date: Dec. 10, 2009

(54) Screening Method

(75) Inventors: Christian Lemmen, Lohmar (DE); Matthias Rarey, Pinneberg (DE); Ingo Schellhammer, Hamburg (DE)

Correspondence Address: LERNER GREENBERG STEMER LLP, P O BOX 2480, HOLLYWOOD, FL, 33022-2480, US

(73) Assignee: BIOSOLVEIT GMBH, St. Augustin (DE)

(21) Appl. No.: 12/158,621

(22) PCT Filed: Dec. 20, 2006
(86) PCT No.: PCT/EP2006/012341

§ 371 Date: Nov. 17, 2008

Related U.S. Application Data
(60) Provisional application No. 60/751,844, filed on Dec. 20, 2005.

(30) Foreign Application Priority Data
Dec. 20, 2005 (DE) 10 2005 061 270.9

(51) Int. Cl. G06F 19/00; G01N 33/48

(52) U.S. Cl. 702/19

(57) ABSTRACT

A method for structure-based virtual screening, wherein compounds for investigation are categorized or sorted into a catalog according to their physicochemical and steric properties. The physicochemical and steric properties of a target are determined, the part(s) of the catalog which match the determined properties of the target are determined, and only the compounds in these parts of the catalog are screened against the target.

The invention relates to a class of methods for structure-based virtual screening by means of database techniques, and, in particular, a sublinear structure-based method for searching for bioactive compounds with the aid of relational databases by means of indexable molecule descriptors.

In general, virtual screening describes the object of searching for small organic molecules (compounds) with biological activity against a specific biological target. In this case, a biological target is a protein, and the biological activity is to inhibit or trigger said protein by means of addition of the compound. This type of screening is described as virtual if it uses a machine-readable description of the compounds and the target and is suited to being carried out automatically, aided by a computer. Here, the exact description of the location of the addition (binding site) is particularly important. The addition is carried out with a specific affinity (binding affinity). The compounds are often highly flexible molecules, which can be in a multiplicity of different forms (conformations). The compounds are represented in the form of molecule graphs with atoms and bonds and further molecule characteristics, if appropriate. Machine-readable descriptions which characterize the chemical characteristics of a molecule are also described as molecule descriptors.

A large number of such descriptions are also known as a virtual library (abbreviated to library).

The description of the target (target descriptor) comprises either the three-dimensional structure of the protein, the three-dimensional structure of at least one biologically active compound, or other three-dimensional descriptions of relevant characteristics for the biological activity. If a three-dimensional structure of the target is available, this is referred to as structure-based virtual screening. The descriptions of compounds and target are in general saved in files in the ASCII format.

Three classes of methods for virtual screening are known:

These are, firstly, three-dimensional search methods based on similarity, by means of which a number of biologically active compounds are analyzed with regard to common physicochemical and structural characteristics (often referred to as pharmacophores). This type of description of the target is used to find compounds with similar characteristics in a library.

A further class comprises alignment-based search methods. Here, one or more three-dimensional structures of biologically active compounds are used as a negative impression of the binding site. The compounds of the library are aligned to this/these structure(s) and their similarity to this description of the target is evaluated with regard to their physicochemical and structural characteristics.

Finally, there are the so-called docking methods which require a three-dimensional model of the binding site of the target. In this case, the compounds are evaluated based on their complementarity to this description of the target with regard to their physicochemical and structural characteristics.

The technical problems and disadvantages of all the methods described above are, on the one hand, their limited precision in the respective calculation and, on the other hand, that they are not suitable, or only suitable to a limited extent, for virtual screening of libraries in a short time and with a high precision. In particular, the alignment and docking-based methods require great computational complexity. A thorough examination of the search space requires rotations and translations of all conformations of the compound and comparison with the target. The more rigorously this search space is examined by a method, the more precisely the geometry of the addition and the binding affinity can be evaluated.

A number of tools for virtual screening are already known. Different screening strategies can be distinguished. Here, a screening strategy refers to a prescription for application of a method or of combinations of a number of methods from the abovementioned classes, coupled with further known algorithmic methods for the purpose of virtual screening of large libraries, if appropriate. In this case, a ranking of potential binding affinity is compiled by means of an evaluation.

In principle, a distinction has to be made in this case between sequential, hierarchical, and filter-based screening strategies. Sequential strategies subject each compound of a library to the same routine comprising one or more of the methods described above. Hierarchical strategies, by contrast, first of all group the compounds of a library and only subject a proportion of them to one or more of the methods described above. Finally, there are the filter-based strategies, which utilize a number of methods for virtual screening one after another. Here, simple and quick methods are firstly applied to all compounds of a library and only a proportion of the compounds, having a higher potential, are subjected to the following, more complex method.

Lemmen, C. and Lengauer, T. “Computational methods for the structural alignment of molecules”, J Comput Aided Mol Des 2000, 14, 215-232 provide an overview of alignment- and similarity-based methods. Even if these approaches use respectively different representations of the compounds and comparison algorithms, the vast majority are sequential and filter-based strategies.

In the following papers, a number of tools for molecular docking, which are used in a number of virtual screening projects, are summarized: Brooijmans, N. and Kuntz, I. D. “Molecular recognition and docking algorithms”, Annu Rev Biophys Biomol Struct 2003, 32, 335-373; Bursulaya, B. D.; Totrov, M.; Abagyan, R. and Brooks, C. L. “3rd Comparative study of several algorithms for flexible ligand docking”, J Comput Aided Mod Des 2003, 17, 755-763; Kellenberger, E.; Rodrigo, J.; Muller, P. and Rognan D. “Comparative evaluation of eight docking tools for docking and virtual screening accuracy”, Proteins 2004, 57, 225-242; and Kitchen, D. B.; Decornez, H.; Furr, J. R. and Bajorath, J. “Docking and scoring in virtual screening for drug discovery: methods and applications”, Nat Rev Drug Discov 2004, 3, 935-949. The majority of these approaches are likewise sequential strategies.

The grouping of the compounds of a library into clusters with similar compounds is a hierarchical strategy. Only one representative of each cluster is then subjected to the screening method. If a representative exhibits a high potential during the process, all compounds of the cluster are subjected to the method.

Cluster methods and classification methods are alternative methods for grouping compounds. Whereas cluster methods comprise a pairwise comparison of all compounds with subsequent aggregation of similar compounds, classification methods utilize physicochemical, topological or structural characteristics in order to divide compounds into pre-specified categories (c.f. van Drie, J. H. and Lajiness, M. S. “Approaches to virtual library design”, Drug Discov Today 1998, 3, 274-283).

In Joseph-McCarthy, D.; Thomas IV, B. E.; Belmarsh, M.; Moustakas, D. and Alvarez, J. C. “Pharmacophore-Based Molecular Docking to Account for Ligand Flexibility”, Proteins 2003, 51, 172-188, and Su, A. I.; Lorber, D. M.; Weston, G. S.; Baase, W. A.; Matthews, B. W. et al. “Docking molecules by families to increase the diversity of hits in database screens: computational strategy and experimental evaluation”, Proteins 2001, 42, 279-293, a further hierarchical strategy is described. First of all, the compounds of a library are preprocessed and divided into groups with similar compounds. Then one representative of each cluster is subjected to a docking method. If a representative is evaluated positively during the process, all compounds of the cluster are docked.

Floriano, W. B.; Vaidehi, N; Zamanakos, G; Goddard III, W. A. “HierVLS, Hierarchical Docking Protocol for Virtual Ligand Screening of Large-Molecule Databases”, J Med Chem 2004, 47, 56-71 describe a filter-based screening strategy. Here, the methods used for the first steps are very fast, but relatively imprecise. The compounds which pass the first set of filters are then subjected to more precise, but slower docking methods.

EP 0 633 534 describes a sequential docking method in which the compounds are arranged at the binding site of the receptor, substantially based on a comparison between the distances of hydrogen bonds in the compound and hydrogen bonds of pseudo-atoms at the binding site of a receptor.

U.S. Pat. No. 6,727,100 substantially follows the approach of imaging compounds on a two-dimensional grid by means of a molecule descriptor in such a way that the distance of the grid points represents the degree of similarity of the corresponding compounds. Furthermore, each grid point is assigned a virtual affinity, a three-dimensional surface is formed over the virtual affinities of each grid point, and the compounds with high virtual affinities are then selected on the basis of the surfaces.

A substantial disadvantage of the methods described above is that all of the compounds (or the representatives of all clusters) have to be processed one after another, and thus require a run-time which increases linearly with the number of compounds in the library.

Furthermore, most of the molecule descriptions represent structural characteristics, in particular atomic coordinates and preferred directions with reference to particular non-covalent bonds between the compound and the target (so-called intramolecular interactions, in this case, for example, hydrogen bonds or interactions of the pi-orbitals). Furthermore, most molecule descriptions represent physicochemical characteristics, in particular the possibilities of forming particular non-covalent bonds between the compound and the target (so-called intermolecular interactions, in this case, for example, hydrogen bonds or interactions of the pi-orbitals). Moreover, steric characteristics, which characterize the spatial extent of the molecule, are introduced into molecule descriptions. Such representations require rotations and translations of a compound within the binding site, which are very complex computationally, in order to evaluate the structural or directionally dependent fit with the receptor.

Therefore, one object on which the invention is based is to provide a structure-based virtual screening method, the average run-time per compound of which is significantly shorter than in the case of known methods.

This object is achieved by a screening method having the features of claim 1.

Thus, according to the invention, a database-supported virtual screening method is created, by means of which the compounds in a library of a database are categorized and sorted in a catalog on the basis of their geometric arrangement of physicochemical and steric characteristics. Furthermore, the corresponding characteristics of a target are used to search for fitting compounds in the database.

A particular advantage of this method is that the compounds in those parts of the catalog which do not fit the target characteristics do not have to be compared to the target at all and hence also do not have to be processed. This results in the run-time of the method according to the invention being able to be substantially decreased. In contrast, in the case of the sequential and filter-based strategies all compounds have to be processed, and even in the case of the hierarchical strategies representatives of all clusters of compounds have to be processed.

A further advantage of the method according to the invention lies in the form of the description used for compounds and target. Both the molecule descriptor and the target descriptor describe physicochemical and steric characteristics in a form in which the complementarity of a compound with the target can be evaluated, without optimization of rotation, translation or conformation of the compound being required. This advantage permits the use of relational database technologies, by means of which the efficiency and scalability of the virtual screening can be improved significantly.

An important advantage of the method according to the invention is finally also that it can be applied not only if the three-dimensional structure of the binding site is known, but it can be expanded in a simple manner so that it can also be applied if the target is defined on the basis of biologically active compounds.

Advantageous developments of the method are contained in the dependent claims.

To carry out this method, at least one descriptor, which is suited to both illustrating the physicochemical and steric characteristics of flexible compounds (molecule descriptor), and to formulating the characteristics of the target (target descriptor), is preferably defined in a format that can be used to query a database.

A substantial characteristic of the descriptor is that it describes both the characteristics of the compounds and the target independently of a (global) coordinate system. In this fashion, the physicochemical and steric characteristics of compounds and target can be directly compared to one another without rotation and translation.

Furthermore, it is preferable if the selectivity of the target descriptor is increased by using directionally dependent conditions and the number of false hits while searching the index of the molecule descriptors is thus decreased.

Overall, these features lead to the result that the run-time of the structure-based virtual screening method is reduced by a few orders of magnitude, while the accuracy can simultaneously be kept at a value which is comparable to that of other approaches.

Further details, features and advantages of the invention emerge from the following description of preferred and exemplary embodiments of the invention on the basis of the drawing, in which:

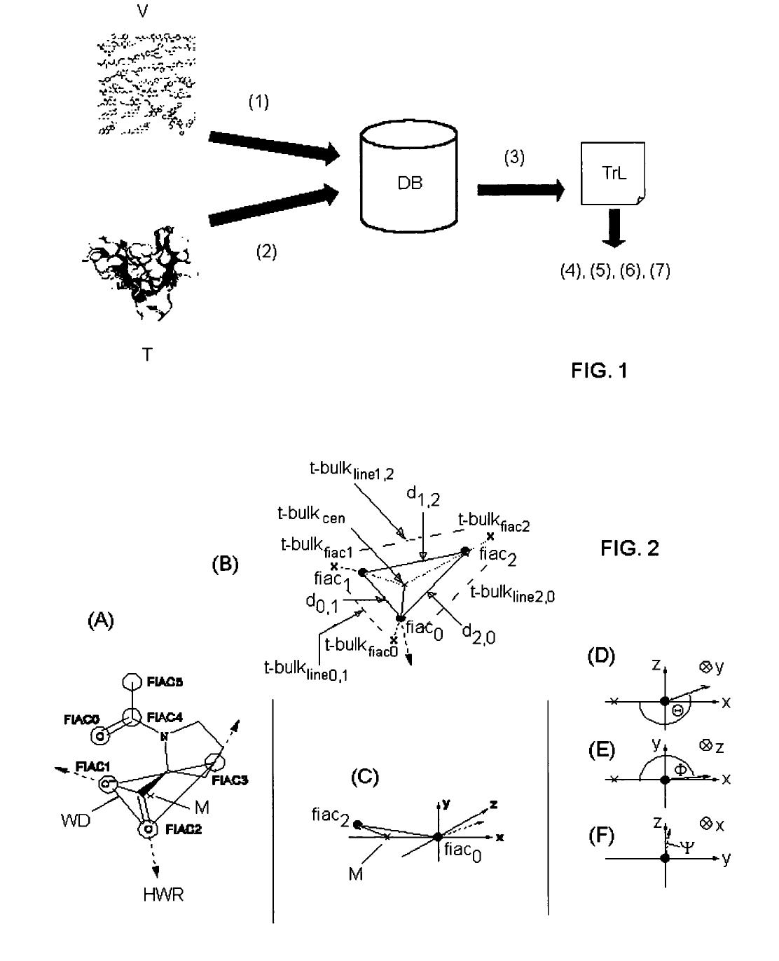

FIG. 1 shows a schematic illustration of fundamental steps of the method;

FIG. 2 shows a schematic illustration of the calculation of molecule descriptors for compounds;

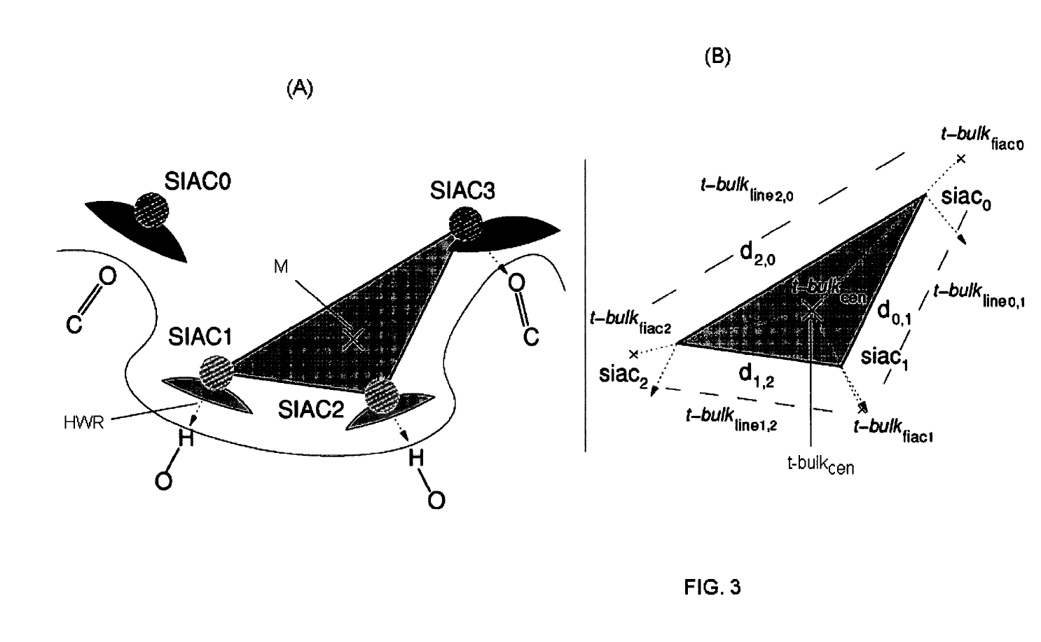

FIG. 3 shows a schematic illustration of the calculation of target descriptors for the target (in this case in the form of a binding site); and

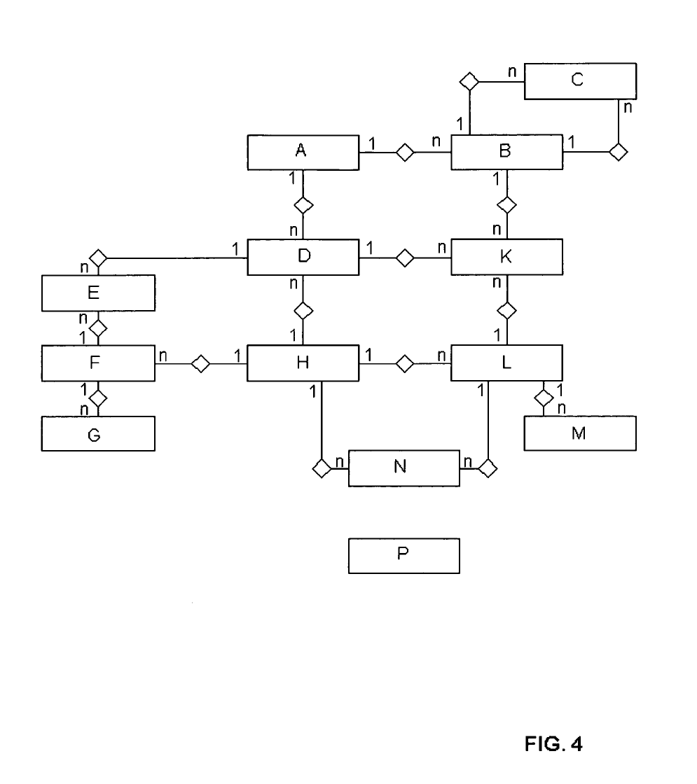

FIG. 4 shows a schematic illustration of a database-structure for carrying out the method.

The embodiment described in the following is based on a target-oriented and catalog-based screening strategy. Using this, the physicochemical and steric characteristics of the target are analyzed and described by one or more target descriptors, by means of which characteristics and conditions in different geometric regions of the target are coded. The method according to the invention uses each of these target descriptors in order to search for compounds in the database or catalog which correspond to the characteristics and conditions of the target descriptor. In this case, the compounds are arranged or categorized in a catalog according to their geometric arrangement of physicochemical and steric characteristics in such a way that only those compounds which are contained in fitting categories of the catalog have to be processed in a search.

It is preferable to use indexable descriptors which also comprise directionally dependent conditions or preferred directions for intermolecular interactions. In particular, a descriptor is used, by means of which physicochemical and/or geometric characteristics of triplets of atoms or atom groups (functional groups) involved in intermolecular interactions, and the molecule shape in the surroundings of the involved atoms (steric property) of both the compound and the target are coded. Thus, in addition to functional groups, their types (based on a conventional categorizing of the interactions) and the distances between pairs of functional groups, their preferred directions are also coded in the indexable descriptor. In this context, indexable means that the descriptors can be sorted according to a number of the characteristic values described above, so that they can be managed for efficient search methods with standardized index structures such as B-trees.

According to the invention, the preferred directions of a functional group in a triplet of functional groups are described by centering a local coordinate system in the functional group and aligning the coordinate system with reference to the other functional groups of the triplet. The preferred direction of a functional group is in this case described relative to the coordinate system, for example by Euler angles with reference to the axes of the local coordinate system.

One point which contributes to a substantial speeding up of the method according to the invention is the application of a relational database technology for saving and looking-up or searching the molecule descriptors. For this reason, it is preferable if all data of the compounds are saved in the tables of a relational database system. Furthermore, database queries based on standard indexes for relational databases are used to look up or search for molecule descriptors which satisfy the query conditions defined by the target (formulated by a set of target descriptors).

While the types of the functional groups of the target and a fitting compound have to be compatible with one another (e.g. donor groups of hydrogen bonds only fit acceptor groups of hydrogen bonds), regions according to the invention for the side lengths of triangles and Euler angles are defined, within which the characteristics of a molecule descriptor are considered to be compatible with the conditions of a target descriptor. It follows that the indexing of the relational database must support region queries.

In the following, the overall procedure of a preferred exemplary embodiment of the method according to the invention is to be explained first of all on the basis of FIG. 1. Here it is assumed that the three-dimensional structure of the binding site of the target is available. However, the method according to the invention can be also used in a simple manner if the target is defined on the basis of biologically active compounds.

In a first preprocessing step (1), to be carried out once at the beginning of the method, the compounds V are first of all split up into smaller pieces or fragments, and all fragments are examined or scanned with regard to their conformations. In the process, functional groups are identified in the compounds and in the fragments which are described as compound interaction centers (CIACs) or fragment interaction centers (FIACs). Optionally the fragmentation can be dispensed with, so that only complete compounds are considered in an analog manner.

Triplets of such FIACs form a fragment interaction triangle for each fragment conformer. The fragment interaction triangles of one or more possible FIAC-triplets code the physicochemical and structural features of a fragment conformer when using the molecule descriptor according to the invention.

According to FIG. 1, the data of the compound, the data of the fragments and the molecule descriptors of the fragment interaction triangles are written into a database DB for the compounds, and are organized by a B-tree, which indexes the FIAC types, the pairwise FIAC distances and the FIAC directions, or each fragment interaction triangle.

In a second step (2), beneficial or advantageous site interaction centers (SIACs) for functional groups of compounds on the binding sites of the receptor or the target T are searched for. Triplets of such SIACs define a set of interaction triangles for positions or sites (the structure of the receptor is assumed to be fixed). The target descriptors of these position interaction triangles code the required FIAC types, the pairwise FIAC distances and the FIAC interaction directions for a fragment, the interaction centers of which fragment are to be aligned to the SIACs of the position interaction triangles or site interaction triangles. In this stage, the database DB of the compounds comprises a table with the conditions of the position interaction triangles or site interaction triangles of the receptor T (set of target descriptors) and a table with fragment interaction triangles of the compounds V (catalog of molecule descriptors).

In a third step (3), all position interaction triangles or site interaction triangles of the receptor are processed, and the conditions of each triangle are translated into an index region query of the table of the fragment interaction triangles of the compounds V, taking a suitable tolerance range in the positive and negative direction into account.

Information about the steric conditions around the interaction triangle is also saved with each descriptor. This holds for both the interaction triangles of the compounds and the interaction triangles of the target. The steric information is already used during the database query to approximately examine whether the fragment of the compound overlaps with the binding site of the receptor. In this manner, an initial test for overlap between the compound and the target is carried out for each hit of the query.

The combination of physicochemical and steric characteristics is of vital importance to the method. Only by means of the assignment of the interaction triangles (physicochemical characteristic) can the relative arrangement of the fragment to the target be described by the alignment of local coordinate systems and the form of the molecule or the target (steric characteristic) can be thus characterized in detail.

On account of the added tolerance ranges, the qualities of these hits (indicated in FIG. 1 in the form of a list of hits TrL) differ in the case of the position interaction triangles and the fragment interaction triangles. For this reason, the quality of each query hit is evaluated with the aid of an evaluation function, and only those hits are saved which lie by a certain amount above a user-defined threshold value.

In a fourth step (4), the algorithm then translates each hit of the query saved in the list of hits TrL into a placing of the fragment conformer or complete molecule on which it is based to the binding site of the receptor. In the process, the alignment of the position interaction triangle with the three FIACs of the fragment interaction triangles defines the rotations and translations of the fragment conformer.

Subsequently, in a fifth step (5), a precise check of the steric fit for each placement of each fragment conformer at the binding site of the receptor is carried out (“overlap test”), and the binding affinity of each placement is estimated, and each placement with a low affinity is discarded.

After the fragments of all query-hits for all position interaction triangles or site interaction triangles have been placed, a sixth step (6) evaluates which fragments belong to which compounds. Furthermore, if appropriate, combinations of placements of different fragments of the same compound that can be realized by a compound conformation are identified. However, this is dispensed with if only complete molecules are saved in the database.

Now, the measure of the affinity of the placed and evaluated fragments is finally used in a seventh step (7) in order to compile a ranking of the compounds that have at least one valid placement.

In the following, the calculation of a molecule descriptor for fragment interaction triangles of the compounds V is to be described on the basis of FIG. 2. The molecule descriptor codes the types of interaction centers, the distances and the interaction directions of a triangle of the FIACs, and additional information about the steric conditions of the fragment surrounding the triangle.

According to FIG. 2(A), three FIACs (FIAC1, FIAC2, FIAC3) of a fragment span an interaction triangle WD. In the illustration, the main interaction direction of the FIAC 2 is designated HWR and the center point of the interaction triangle is designated M.

According to FIG. 2(B), a canonical arrangement algorithm then sorts the three FIACs (fiac₀, fiac₁, fiac₂) in such a way that the types of the FIACs (t₀, t₁, t₂) and the lengths of their adjoining triangle sides (d_{0,1}, d_{1,2}, d_{2,0}) are arranged in a lexicographical sequence (t₀, d_{0,1})≦_{L} (t₁, d_{1,2})≦_{L} (t₂, d_{2,0}).

The corners of the triangle (the FIACS) describe a plane in which the triangle lies. On the basis of the sorting of the three FIACs, the space above and the space below the plane of the triangle can be differentiated unambiguously. In order to describe the location of the steric mass of the fragment, copies of the sides of the triangle are displaced outward within the plane (away from the center of the triangle) and then shifted upward and downward. This results in altogether three lines above (t-bulk_{line0,1}, t-bulk_{line1,2} t-bulk_{line2,0}) the triangle and three lines below (b-bulk_{line0,1}, b-bulk_{line1,2}, b-bulk_{line2,0}). In FIG. 2(B), only the lines above the triangle are illustrated.

Each of these lines is divided into a constant number (nine in this case) of discrete segments of equal length; each segment of each line is represented by one bit in the descriptor. If a segment of the fragment is partially or wholly covered by steric mass, the bit in the descriptor is set, otherwise it is not set. Preferably 27 bits are respectively used for the region above the triangle and the region below the triangle.

Thus, using this, a bit string codes the existence (e.g. the bit of the line segment is set) or the lack (the bit is not set) of steric mass of the compound along every line. As an alternative, it is also possible to measure distances from the coordinate origin to the surface of the molecule in different directions.

As is illustrated in FIG. 2(B), three further bits are used in each case above and below the triangle to code the existence of steric mass at the vertices (displaced upward and downward in the same manner) of the triangle (t-bulk_{fiac0}, t-bulk_{fiac1}, t-bulk_{fiac2} and b-bulk_{fiac0}, b-bulk_{fiac1}, b-bulk_{fiac2}). Furthermore, in each case one bit is used to code the existence of steric mass above and below the center of the triangle (t-bulk_{cen} and b-bulk_{cen}).

In order to describe the direction of the interaction of a FIAC according to FIG. 2(C), the next step is to arrange the triangle in a local coordinate system such that the FIAC coincides with the origin, the center of the triangle lies on the negative x-axis and the FIAC following in the canonical order lies in the x-z plane with a negative x-value.

According to the FIGS. 2(D) to 2(F), the direction of the interaction can now be described by three Euler angles:

According to FIG. 2(D), θ represents the angle between the negative x-axis and the direction of the interaction projected onto the x-z plane.

According to FIG. 2(E), φ represents the angle between the negative x-axis and the direction of the interaction projected onto the x-y plane.

Finally, according to FIG. 2(F), ψ designates the angle between the positive z-axis and the direction of the interaction projected onto the x-y plane.

According to FIGS. 3(A) and 3(B), the generation of the target descriptors of the position interaction triangles or site interaction triangles of the SIACs of the receptors is carried out in an analogous manner, as described above with reference to the FIGS. 2(A) and 2(B) and with similar or corresponding vertices, sides, lines, etc. being provided with the same or a corresponding designation, so that in this respect a renewed description can be dispensed with. The target descriptor codes the types of the interaction directions HWR of a triangle of the SIACs and additional information about the steric mass of the receptor surrounding the triangle.

In this case, a triplet of the SIACs defines a position interaction triangle or site interaction triangle which can be described by the same descriptor which is used for the fragment interaction triangles. With regard to determining the direction of the interaction HWR, FIGS. 2(C) to 2(F) apply correspondingly.

As has already been explained, the method according to the invention is preferably carried out with the aid of a relational database. The data of the compounds and the fragments are in this case saved in tables of the database. FIG. 4 schematically shows the structure of such a database in the form of its tables and their relative links. Using index structures according to the B-tree type, access to these tables and the query of the tables can be significantly sped up.

All attributes of the fragment triangles with the exception of the bit strings of the steric mass are contained in the B-tree index of the largest and most queried table “geometries of the fragment interaction triangles” (Table N).

In detail, the tables in FIG. 4 comprise the following data:
A the compounds,
B the compound interaction centers (CIACs),
C the CIAC distances,
D the links to contained fragments of a compound,
E the atomic reference coordinates of the active fragment conformer for a particular compound,
F the atoms of a fragment,
G the atomic coordinates of all fragment conformers,
H the fragments,
K the links of CIACs to the corresponding FIACs of the contained fragments,
L the FIACs,
M the FIAC coordinates of all fragment conformers,
N the geometries of the fragment interaction triangles of the FIACs, and
P the geometries of the position interaction triangles or site interaction triangles of the SIACs.

The method was tested with different virtual screening trials on nine target proteins with pharmaceutical relevance. The results were compared to the results of a known molecule docking program (FlexX).

In the process it was shown that in six of eleven cases the method according to the invention exhibited similar or slightly improved performance compared to the FlexX program, whereas in three cases it was worse than the known FlexX program. Using the screening method according to the invention, the average run-time per compound could be reduced by a factor of between 10 and 60 in comparison to the FlexX program. It can be inferred from the literature that FlexX is one of the fastest docking programs currently available. Furthermore, it could be shown that the average run-time per compound decreased with an increasing size of the library, that is to say with an increasing number of compounds, when using the method according to the invention.

1-24. (canceled)

25. A method for structure-based virtual screening of bioactive compounds, comprising the following steps:
sorting compounds to be searched into a catalog according to a geometric arrangement of their physicochemical and steric characteristics;
calculating and saving a compound descriptor for the indexable description of the geometric arrangement of the physicochemical and steric characteristics of the compounds;
determining the geometric arrangement of physicochemical and steric characteristics of a target, and calculating a target descriptor for the indexable description of the geometric arrangement of physicochemical and steric characteristics of the target;
determining at least parts of the catalog which fit the determined characteristics of the target by indexing access of the compounds of the catalog using the compound and target descriptors; and
screening the compounds in said parts of the catalog relative to the target.

26. The method according to claim 25, wherein the target has characteristics which represent structure of a binding site of a receptor, or a relative orientation of known compounds, that are active with respect to one of the target or a biological activity of the target.

27. The method according to claim 25, including the step of describing at least one target or molecular descriptor by the geometric arrangement of physicochemical and steric characteristics of at least one of the target and the compounds.

28. The method according to claim 27, including the steps of indexing the descriptors for sorting according to their characteristic values and managing the descriptors with standardized index structures such as B-trees.

29. The method according to claim 27, including the step describing the descriptors by directionally dependent conditions or preferred directions with reference to atomic coordinates.

30. The method according to claim 27, including the step of describing the descriptors by physicochemical and steric characteristics of triplets of functional groups of at least one of the compounds and target.

31. The method according to claim 30, including the step of describing the descriptors by directionally dependent conditions or preferred directions of functional groups of at least one of the compounds and target with reference to atomic coordinates.

32. The method according to claim 31, including the step of describing preferred directions of a functional group in a triplet of functional groups by centering a local coordinate system in the functional group and aligning the coordinate system with reference to the other functional groups of the triplet.

33. The method according to claim 31, including the step of describing a preferred direction of a functional group relative to a local coordinate system by Euler angles with reference to the axes of the local coordinate system.

34. The method according to claim 33, including the step of defining regions for the side lengths of triangles and Euler angles where the characteristics of a compound descriptor are considered complementary to conditions of a target descriptor.

35. The method according to claim 25, including a first step of splitting the compounds into one or more pieces and examining them with regard to their conformation to identify the relative spatial position of functional groups in the compounds or the fragments and spatial position of compound interaction centers or fragment interaction centers.

36. The method according to claim 35, including the steps of forming one or more fragment interaction triangles for each fragment conformer by triplets of FIACs, and using a molecule descriptor so that the fragment interaction triangles describe physicochemical and steric features of a fragment conformer.

37. The method according to claim 35, including a second step of searching advantageous binding site interaction centers of the receptor or the target for functional groups of the compounds.

38. The method according to claim 37, including the steps of defining a set of interaction triangles for positions or sites of the target by triplets of SIACs, and describing the descriptors of the interaction triangles for required FIAC types, pairwise FIAC distances and FIAC interaction directions for a fragment, and aligning the interaction centers of the fragment relative to the SIACs of the interaction triangles of the target.

39. The method according to claim 38, including a third step of processing all position interaction triangles or site interaction triangles of the receptor, and translating the conditions of each interaction triangle of the target into an index region query of a table of the fragment interaction triangles of the compounds, taking into account a suitable tolerance range in the positive and negative direction.

40. The method according to claim 39, including a fourth step of translating by using an algorithm each hit of the query saved in a list of hits into a placement of the fragment conformer or complete molecule on which it is based to the binding site of the receptor.

41. The method according to claim 40, including a fifth step of checking the steric fit for each placement of each fragment conformer at the binding site of the receptor and estimating the binding affinity of each placement, and discarding placements with a low affinity.

42. The method according to claim 41, including a sixth step of evaluating fragments that belong compounds and identifying applicable combinations of placements of different fragments of the same compound that can be realized by a compound conformation.

43. The method according to claim 42, including a seventh step of measuring the affinity of placed and evaluated fragments for compiling a ranking of the compounds that have at least one valid placement.

44. The method according to claim 25, including the step of saving the catalog in a virtual library of a database of a computer program.

45. The method according to claim 27, including the step of defining at least one of the target descriptors for querying the catalog saved in a virtual library of a database of a computer program.

46. A computer program comprising a program code for carrying out the method according to claim 25 if run on a computer device.

47. The computer program according to claim 46, including the step of saving the data of the compounds and the fragments in tables of a relational database.

48. A computer program product saved on a computer-readable medium, comprising a program code for carrying out the method according to claim 25 if run on a computer device.
